(19) United States
(12) Patent Application Publication
(10) Pub. No.: US 2005/0191247 A1
(43) Pub. Date: Sep. 1, 2005

(54) Chlorhexidine compositions

(76) Inventors: David Drake, Iowa City, IA (US); Cindy L. Marek, Iowa City, IA (US)

Correspondence Address: Schwegman, Lundberg, Woessner & Kluth, P.A., P.O. Box 2938, Minneapolis, MN, 55402, US

(21) Appl. No.: 11/069,576

(22) Filed: Mar. 1, 2005

Related U.S. Application Data
(60) Provisional application No. 60/549,224, filed on Mar. 1, 2004.

(51) Int. Cl. A61K 31/205; A61K 7/16; A61K 7/18

(52) U.S. Cl. 424/49; 514/554

(57) ABSTRACT

The invention provides alcohol-free chlorhexidine compositions, improved methods for preparing colloidal chlorhexidine containing compositions, and methods for promoting an antibacterial effect comprising administering chlorhexidine compositions to the mouth of an animal. The invention also provides an improved chlorhexidine composition comprising a flavoring agent (e.g. a raspberry flavoring agent) that increases the antibacterial activity of the composition.

### CLAIM OF PRIORITY

Benefit of priority is hereby claimed to U.S. Provisional Patent Application Ser. No. 60/549,224, filed on Mar. 1, 2004, which application is herein incorporated by reference.

### FIELD OF THE INVENTION

The invention relates generally to oral antibacterial compositions containing chlorhexidine and to methods useful for the preparation and use of such compositions.

### BACKGROUND

Chlorhexidine is recognized as a “gold standard” antibacterial agent and has been extensively studied. Oral chlorhexidine compositions have been used to reduce levels of cariogenic bacteria in order to prevent dental caries and improve oral hygiene. See Beighton et al., J. Clin. Periodontol., 18(2):85 (1991); Bowden, J Can. Dent. Assoc., 62(9):700 (1996); Grytten et al., Caries Res., 22(6):371 (1988); Grytten et al., Acta Odontol. Scand., 45(6):429 (1987); Marsh, J Applied Bacter., 81(2): 120 (1996); Moore et al., J Periodontal, 60(2):78 (1989); Tenovuo et al., Caries Res., 26(4):275 (1992); Ullsfoss et al., Scand. J Dent. Res., 102(2):109 (1994); Van der Hoeven and Schaeken, Caries Res., 29(2):159 (1995); Waerhaug et al., J. Clin. Periodontol., 11(3):176 (1984); Waler and Rolla, Scand. J Dent. Res., 90(2):131 (1982); and Wilson et al., Oral Microbial. Immunol., 11:188 (1996).

Dental caries is an infectious disease caused by cariogenic bacteria, in particular Streptococcus mutans. Chemotherapeutic agents that target Streptococcus mutans have been recognized for their ability to prevent dental caries. A number of studies have demonstrated statistically significant reduction in S. mutans colonization in children and adults after daily or interval use of chlorhexidine compositions. Such compositions include rinses, varnishes, and gels. The outcomes and efficacy of the chlorhexidine compositions in caries prevention has been comprehensively reviewed. See Kidd, International Dent. J, 41:279; Emilson, J Dent. Res., 73(3):682 (1994); Van Rijkom et al, J Dent. Res., 75(2):790 (1996); and Ismail, Comm. Dent. Oral Epidemiol., 26:49-61(1998).

Oral chlorhexidine treatments, however, suffer from several drawbacks. For example, it is well known that chlorhexidine products have an extremely bitter taste. This objectionable taste causes major compliance problems, particularly with pediatric patients. In order to combat the bitter taste, certain flavoring agents and sweetening agents have been added to chlorhexidine formulations.

Additionally, chlorhexidine is a strong basic material that reacts with a wide variety of compounds and chemical structures that are often used in the commercial production of chlorhexidine products. Many of these reactions result in reduced antibacterial activity of chlorhexidine. Thus, the addition of many flavoring agents, which are often aldehydes in structure, or sweetening agents can reduce or eliminate chlorhexidine's antibacterial activity via chemical reactions that include salt formation and precipitation. For example, the sweetener used in commercial chlorhexidine rinses (Chlorhexidine Gluconate Oral Rinse, Alpharma) in the United States is sodium saccharin. Saccharin has been shown to reduce antibacterial activity of chlorhexidine gels. See Cury, et al., Braz. Dent. J, 11: 29-34 (2000).

Furthermore, commercial chlorhexidine compositions typically contain alcohol as a solvent. Alcohol can cause burning in the mouth, particularly if the alcohol contacts open sores. It is also possible that repeated use of alcohol containing compositions may cause sores in the mouth. Xerostomia (dry mouth) is a common oral condition that predisposes patients to an increased rate of intraoral infections and dental caries. Because alcohol has a strong drying effect in the mouth, the symptoms of xerostomia are worsened by alcohol containing products.

Accordingly, chlorhexidine oral compositions that overcome one or more of the problems associated with existing compositions would be valuable, for example, to patients in need of chlorhexidine treatments.

### SUMMARY OF CERTAIN EMBODIMENTS OF THE INVENTION

Alcohol-free chlorhexidine compositions that are useful to promote an antibacterial effect in the mouth of an animal are described herein. Improved methods for preparing hydrocolloidal chlorhexidine compositions are also described herein. Accordingly, certain embodiments of the invention provide an alcohol-free hydrocolloidal composition comprising chlorhexidine. The lack of alcohol in the composition helps assure that the composition will not cause burning symptoms or enhance xerostomia when applied to the mouth of an animal.

Certain embodiments of the invention also provide a hydrocolloidal composition comprising chlorhexidine and a flavoring agent that does not significantly reduce the antibacterial activity of the composition.

Certain embodiments of the invention also provide a hydrocolloidal composition comprising chlorhexidine and a sweetening agent that does not significantly reduce the antibacterial activity of the oral composition.

Certain embodiments of the invention also provide a hydrocolloidal composition comprising chlorhexidine and a flavoring agent that increases the antibacterial activity of the oral composition.

Certain embodiments of the invention also provide improved methods for preparing chlorhexidine compositions, and in particular, alcohol free chlorhexidine compositions. It has been determined that by changing the order of mixing, one can improve the process of preparing chlorhexidine compositions. It has also been determined that the processes of the invention provide improved chlorhexidine oral compositions in terms of yield and/or performance as an antibacterial composition. Accordingly, certain embodiments of the invention also provide a method for preparing an oral composition containing chlorhexidine comprising adding a solution containing chlorhexidine or a salt thereof to a hydrocolloidal suspension (e.g. using geometric dilution).

Certain embodiments of the invention also provide a method for preparing an oral composition comprising chlorhexidine comprising adding raspberry flavor to an oral composition comprising chlorhexidine.

Certain embodiments of the invention also provide a method comprising mixing a sweetening agent into heated water (≦60° C.) with stirring to enhance dissolution of the sweetening agent; allowing the mixture to cool to about room temperature (e.g., about 20-25° C./68-77° F.) with continued stirring; slowly adding hydroxypropyl methylcellulose (HPMC) to the mixture with stirring; slowly (e.g. dropwise); adding a solution of chlorhexidine diluted to less than 10% chlorhexidine by weight to the HPMC mixture by geometric dilution with stirring to provide a chlorhexidine mixture; optionally adding a copper salt, a zinc salt, or a combination thereof, with stirring to the chlorhexidine mixture; adding a raspberry flavoring agent with stirring to the chlorhexidine mixture; and diluting with sterile water for irrigation to produce an oral composition containing chlorhexidine.

Certain embodiments of the invention also provide a method to promote an antibacterial effect comprising administering a composition of the invention to the mouth of an animal.

Certain embodiments of the invention also provide a method to promote an antibacterial effect comprising administering an alcohol-free oral composition comprising chlorhexidine to the mouth of an animal.

Certain embodiments of the invention also provide a method to promote an antibacterial effect comprising administering an oral composition comprising chlorhexidine and a sweetening agent that does not significantly decrease the antibacterial activity of the composition to the mouth of an animal.

Certain embodiments of the invention also provide a method to promote an antibacterial effect comprising administering to an animal an oral composition comprising chlorhexidine and a flavoring agent that does not significantly reduce the antibacterial activity of the composition.

Certain embodiments of the invention also provide a method to promote an antibacterial effect comprising administering to an animal an oral composition comprising chlorhexidine and a flavoring agent that increases the antibacterial activity of the composition.

Certain embodiments of the invention also provide a method to promote an antibacterial effect comprising administering to an animal an oral composition comprising chlorhexidine and a raspberry flavoring agent that increases the antibacterial activity of the composition.

Certain embodiments of the invention also provide novel compositions and intermediates disclosed herein that are useful for preparing chlorhexidine compositions.

Certain embodiments of the invention also provide composition as described herein for use in medical therapy.

Certain embodiments of the invention also provide the use of a composition as described herein to prepare a medicament useful to promote to an antibacterial effect in an animal. In some embodiments, the animal is a human.

Certain embodiments of the invention also provide the use of a composition as described herein to prepare a medicament useful to prevent or treat dental caries in an animal. In some embodiments, the animal is a human.

### BRIEF DESCRIPTION OF THE FIGURES

FIG. 1 depicts the results of experiments examining the activity of chlorhexidine and chlorhexidine-cation combinations.

FIG. 2 depicts the results of experiments examining the activity of chlorhexidine and chlorhexidine-flavoring agent combinations.

FIG. 3 depicts the results of experiments examining the effect of chlorhexidine compositions of the ability of S. mutans to produce acid.

### DETAILED DESCRIPTION OF EXEMPLARY EMBODIMENTS

Certain embodiments of the invention provide an alcohol-free hydrocolloidal chlorhexidine composition. In certain embodiments, the composition comprises chlorhexidine gluconate. In certain embodiments, the composition comprises chlorhexidine diacetate or chlorhexidine dihydrochloride. In certain embodiments, the composition comprises a sweetening agent. In certain embodiments, the sweetening agent does not significantly reduce the antibacterial activity of the composition. In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the composition comprises a flavoring agent. In certain embodiments, the flavoring agent is a raspberry flavoring agent. In certain embodiments, the composition comprises a copper salt, a zinc salt, or a combination thereof. In certain embodiments, the copper and/or zinc ion is present in at least about 0.05% w/w. In certain embodiments, the composition comprises cellulose or a cellulose-derivative. In certain embodiments, the composition comprises hydroxypropyl methylcellulose, hydroxyethyl cellulose, or hydroxypropyl cellulose, or a combination thereof. In certain embodiments, the composition comprises hydroxypropyl methylcellulose.

Certain embodiments of the invention provide a hydrocolloidal composition comprising chlorhexidine and a flavoring agent that does not significantly reduce the antibacterial activity of the composition. In certain embodiments, the composition comprises chlorhexidine gluconate. In certain embodiments, the composition comprises chlorhexidine diacetate or chlorhexidine dihydrochloride. In certain embodiments, the composition comprises a sweetening agent. In certain embodiments, the sweetening agent does not significantly reduce the antibacterial activity of the composition. In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the flavoring agent is a raspberry flavoring agent. In certain embodiments, the composition comprises a copper salt, a zinc salt, or a combination thereof. In certain embodiments, the copper and/or zinc ion is present in at least about 0.05% w/w. In certain embodiments, the composition comprises cellulose or a cellulose-derivative. In certain embodiments, the composition comprises hydroxypropyl methylcellulose, hydroxyethyl cellulose, or hydroxypropyl cellulose, or a combination thereof. In certain embodiments, the composition comprises hydroxypropyl methylcellulose.

Certain embodiments of the invention provide a hydrocolloidal composition comprising chlorhexidine and a sweetening agent that does not significantly reduce the antibacterial activity of the oral composition. In certain embodiments, the composition comprises chlorhexidine gluconate. In certain embodiments, the composition comprises chlorhexidine diacetate or chlorhexidine dihydrochloride. In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the composition comprises a flavoring agent. In certain embodiments, the flavoring agent is a raspberry flavoring agent. In certain embodiments, the composition comprises a copper salt, a zinc salt, or a combination thereof. In certain embodiments, the composition does not comprise a surfactant. In certain embodiments, the composition comprises cellulose or a cellulose-derivative. In certain embodiments, the composition comprises hydroxypropyl methylcellulose, hydroxyethyl cellulose, or hydroxypropyl cellulose, or a combination thereof. In certain embodiments, the composition comprises hydroxypropyl methylcellulose.

Certain embodiments of the invention provide an oral composition comprising chlorhexidine and a flavoring agent that increases the antibacterial activity of the oral composition. In certain embodiments, the composition comprises chlorhexidine gluconate. In certain embodiments, the composition comprises chlorhexidine diacetate or chlorhexidine dihydrochloride. In certain embodiments, the composition comprises a sweetening agent. In certain embodiments, the sweetening agent is not sorbitol. In certain embodiments, the sweetening agent is selected from the group consisting of aspartame, sorbitol, xylitol, and sucralose. In certain embodiments, the sweetening agent is aspartame. In certain embodiments, the composition is a hydrocolloidal composition. In certain embodiments, the flavoring agent is a raspberry flavoring agent. In certain embodiments, the composition comprises a copper salt, a zinc salt, or a combination thereof. In certain embodiments, the copper and/or zinc ion is present in at least about 0.05% w/w. In certain embodiments, the composition comprises cellulose or a cellulose-derivative. In certain embodiments, the composition comprises hydroxypropyl methylcellulose, hydroxyethyl cellulose, or hydroxypropyl cellulose, or a combination thereof. In certain embodiments, the composition comprises hydroxypropyl methylcellulose.

Certain embodiments of the invention provide a method for preparing a hydrocolloidal chlorhexidine composition comprising adding a solution containing chlorhexidine or a salt thereof to a hydrocolloidal suspension to provide the hydrocolloidal chlorhexidine composition. In certain embodiments, the hydrocolloidal chlorhexidine composition is alcohol-free. In certain embodiments, the solution containing chlorhexidine comprises water. In certain embodiments, the solution containing chlorhexidine is chlorhexidine in water. In certain embodiments, the solution containing chlorhexidine is added to the hydrocolloidal suspension dropwise. In certain embodiments, the solution containing chlorhexidine and the hydrocolloidal suspension are combined by geometric dilution. In certain embodiments, the hydrocolloidal suspension is prepared from water and one or more suspending agents. In certain embodiments, the hydrocolloidal suspension that comprises chlorhexidine gluconate. In certain embodiments, the hydrocolloidal suspension comprises chlorhexidine diacetate or chlorhexidine dihydrochloride. In certain embodiments, the hydrocolloidal suspension comprises a sweetening agent. In certain embodiments, the sweetening agent does not significantly reduce the antibacterial activity of the composition. In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the method further comprises adding a flavoring agent to the hydrocolloidal chlorhexidine composition. In certain embodiments, the flavoring agent is a raspberry flavoring agent. In certain embodiments, the method further comprises adding a copper salt, a zinc salt, or a combination thereof, to the hydrocolloidal chlorhexidine composition. In certain embodiments, at least about 0.05% w/w of copper and/or zinc ion is added to the hydrocolloidal chlorhexidine composition. In certain embodiments, the hydrocolloidal suspension comprises cellulose or a cellulose-derivative. In certain embodiments, the hydrocolloidal suspension comprises hydroxypropyl methylcellulose, hydroxyethyl cellulose, or hydroxypropyl cellulose, or a combination thereof. In certain embodiments, the hydrocolloidal suspension comprises hydroxypropyl methylcellulose.

Certain embodiments of the invention provide a method for preparing an improved composition comprising chlorhexidine comprising combining a flavoring agent that increases the antibacterial activity with a composition comprising chlorhexidine. In certain embodiments, the flavoring agent is a raspberry flavoring agent. In certain embodiments, the composition comprising chlorhexidine is formulated for oral administration to an animal. In certain embodiments, the composition comprising chlorhexidine is a hydrocolloidal composition. In certain embodiments, the composition comprising chlorhexidine is alcohol-free. In certain embodiments, the improved composition comprising chlorhexidine is alcohol-free. In certain embodiments, the composition comprising chlorhexidine comprises chlorhexidine gluconate. In certain embodiments, the composition comprising chlorhexidine comprises chlorhexidine diacetate or chlorhexidine dihydrochloride. In certain embodiments, the composition comprising chlorhexidine is a hydrocolloidal suspension comprising one or more suspending agents. In certain embodiments, the composition comprising chlorhexidine comprises a sweetening agent. In certain embodiments, the sweetening agent does not significantly reduce the antibacterial activity of the composition. In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the improved composition comprising chlorhexidine comprises a copper salt, a zinc salt, or a combination thereof. In certain embodiments, the improved composition comprising chlorhexidine comprises at least about 0.05% w/w copper and/or zinc ions. In certain embodiments, the composition comprising chlorhexidine comprises cellulose or a cellulose-derivative. In certain embodiments, the composition comprising chlorhexidine comprises hydroxypropyl methylcellulose, hydroxyethyl cellulose, or hydroxypropyl cellulose, or a combination thereof. In certain embodiments, the composition comprising chlorhexidine comprises hydroxypropyl methylcellulose.

Certain embodiments of the invention provide a method comprising:
mixing a sweetening agent into heated water at about 50° C. with stirring to provide a mixture;
allowing the mixture to cool to about 20-25° C.;
slowly adding hydroxypropyl methylcellulose to the mixture with stirring;
adding a solution of chlorhexidine to the mixture dropwise by geometric dilution with stirring to provide a chlorhexidine mixture;
optionally adding a copper salt, a zinc salt, or a combination thereof, with stirring to the chlorhexidine mixture;
optionally adding a raspberry flavoring agent with stirring to the chlorhexidine mixture; and
optionally diluting the mixture with sterile water;
to produce an oral composition containing chlorhexidine.
   In certain embodiments, the sweetening agent is selected from the group consisting of aspartame, sorbitol, xylitol, and sucralose. In certain embodiments, the sweetening agent is aspartame. In certain embodiments, the sweetening agent is not sorbitol. In certain embodiments, the aqueous solution of chlorhexidine is added to the mixture at a rate of about one drop per second.

Certain embodiments of the invention provide a composition prepared by a method described herein.

Certain embodiments of the invention provide method to promote an antibacterial effect comprising administering an effective amount of a composition as described herein to an animal. In certain embodiments, the composition is administered to the mouth of the animal.

Certain embodiments of the invention provide a method to prevent or treat dental caries comprising administering an effective amount of a composition as described herein to the mouth of an animal.

Certain embodiments of the invention provide a composition as described herein for use in medical therapy.

Certain embodiments of the invention provide the use of a composition as described herein to prepare a medicament useful to promote to an antibacterial effect in an animal. In certain embodiments, the animal is a human.

Certain embodiments of the invention provide the use of a composition as described herein to prepare a medicament useful to prevent or treat dental caries in an animal. In certain embodiments, the animal is a human.

Unless stated otherwise, the following terms and phrases as used herein are intended to have the following meanings.

“Alcohol-free” typically means that the composition contains less than about 10% alcohol by weight. In certain specific embodiments of the invention alcohol free can also mean that the composition contains less than about 5%, about 2%, or about 1% alcohol by volume. Preferably, alcohol free means that the composition contains less than about 0.5% alcohol by volume. More preferably, alcohol free means that the composition contains no alcohol.

A “bitter blocking agent” is a compound that reduces or eliminates the body's ability to taste bitterness. Addition of these compounds to inherently bitter substances such as chlorhexidine improves palatability and patient compliance. One preferred bitter blocking agent is adenosine monophosphate (AMP). Other chemical entities, such as monosodium glutamate (MSG) and table salt, have also been used in an attempt to mask bitter flavor. (Pszczola, D., Ten Ingredient Developments That May Impact the Future of Foods, Food Technology, 57(7), 76-88).

A “hydrocolloidal mixture” or “hydrocolloidal composition” as used herein includes a colloid wherein a liquid (e.g. water) is dispersed in a solid continuous phase. These terms also include a liquid continuous phase containing a dispersed solid, also know as a sol. An example of a hydrocolloidal mixture or hydrocolloidal composition is a hydrated solid material that forms a semi-solid mixture. Preferable hydrocolloidal gels are made from suspending agents that are hydrophilic, non-ionic polymers. These viscosity producing agents typically crosslink with water upon hydration and form “bridges” with the attracted water molecules to give consistency to the hydrogel. The non-ionic nature of these polymers allows for the incorporation of electrolytes and provides stability over a wide range of pH.

As used herein, the phrase “does not significantly decrease the activity of the oral composition” typically means that the activity of the composition with a particular additive is decreased less than about 10% compared to the activity of the composition without that additive. In one specific embodiment of the invention, the phrase “does not significantly decrease the activity of the oral composition” means that the activity of the composition with a particular additive is decreased less than about 5% compared to the activity of the composition without that additive. In another specific embodiment of the invention, the phrase “does not significantly decrease the activity of the oral composition” means that the activity of the composition with a particular additive is decreased less than about 2% compared to the activity of the composition without that additive. In a preferred embodiment of the invention, the phrase “does not significantly decrease the activity of the oral composition” means that the activity of the composition with a particular additive is the same, or greater, than the activity of the composition without that additive.

The term “oral composition” includes rinses, mouthwashes, gels, pastes, and varnishes.

Suspending Agents

Suspending agents that can be used to prepare chlorhexidine colloidal mixtures with water include non-ionic hydrophilic polymers. These polymers typically crosslink with water upon hydration and form “bridges” with the attracted water molecules to give consistency to the gel. In one embodiment of the invention, the polymers are non-ionic in nature. This will help to avoid any undesired chemical reactions with the chlorhexidine product. Acceptable non-ionic cellulose ethers include, for example, hydroxypropyl methylcellulose (HPMC), hydroxyethyl cellulose, and hydroxypropyl cellulose. Other predominantly non-ionic water soluble suspending agents are also acceptable.

HPMC is soluble in water and some organic solvents and is available in a wide variety of cps ranging from 5 to 100,000. Solubility varies with viscosity: the lower the viscosity is, the higher the solubility is. The lower methoxyl content in HPMC, the higher gelatification temperature, lower solubility in water and surface activity. The cloud point of HPMC (the temperature at which it becomes insoluble in water) is 50° C. The preferred temperature for hydration is 25° C.

In one embodiment, hydroxypropyl methylcellulose 2208 has a viscosity of about 4000 cps (range about 3000 to about 5600 cps measured as a 2 weight percent aqueous solution at 20° C.).

Sweetening Agents

Non-cariogenic sweeteners are preferred in the development of oral chlorhexidine products. Chlorhexidine is a chemically reactive material that can combine with other reactive chemical compounds, including sweetening agents, to form complexes that render the chlorhexidine less effective as an antimicrobial agent. Sodium saccharin, a common non-cariogenic sweetener used in commercial chlorhexidine products (Chlorhexidine Gluconate Oral Rinse, Alpharma) has been shown to reduce antibacterial activity of chlorhexidine gels. See Cury, et al., Braz. Dent. J, 11: 29-34 (2000).

Accordingly, in one embodiment, the compositions of the invention comprise one or more sweetening agents that do not significantly decrease the antibacterial activity of the oral composition. One such sweetener is aspartame.

Aspartame is a dipeptide of two amino acids that is approximately 200 times sweeter than table sugar. Aspartame's potency varies depending on application and concentration. Higher potencies are reached at lower concentrations. Typical beverage concentrations of aspartame range from 0.01% to 0.2%. In water, the solubility of aspartame is approximately 1% at 20° C. and 3% at 50° C. Agitation and increased temperature (up to 60° C.) accelerate dissolution. The dissolution time in water depends upon initial particle size (granular or powder) and varies from 5-30 minutes. Solubility of aspartame is enhanced in acidic solutions. Aspartame is insoluble in alcohol.

Stability of aspartame in liquid vehicles is influenced by temperature and acidity (pH). Optimal stability is typically achieved at a pH of about 4.2. Typically aspartame degrades into its individual constituent amino acids in liquid systems with alkaline pH values (pH.≧7).

In some embodiments of the invention, the concentration (W/W) of a sweetening agent, e.g., aspartame, is within the range of about 20%-150% of a concentration of a sweetening agent used herein. In some embodiments of the invention, the concentration (W/W) of the sweetening agent is about 50% of a concentration of a sweetening agent used herein. In some embodiments of the invention, the concentration (W/W) of the sweetening agent is within the range of about 50%-100% of a concentration of a sweetening agent used herein. For example, in some embodiments of the invention, the concentration (W/W) of the sweetening agent is about 0.4%; in some embodiments, the concentration (W/W) of the sweetening agent is about 0.2%; in some embodiments the concentration (W/W) of the sweetening agent is within the range of from about 0.08% to about 0.6%; in some embodiments, the concentration (W/W) of the sweetening agent is within the range of from about 0.2% to about 0.4%.

Other suitable sweetening agents include sorbitol, xylitol, and sucralose. In one embodiment, the compositions of the invention do not include sorbitol. In another embodiment, the methods of the invention exclude the administration of compositions that include sorbitol.

In some embodiments of the invention, the sweetening agent includes any one of the following sweetening agents, or combinations thereof: saccharin (e.g., sodium saccharin), sucralose, stevia (e.g., Stevia rebaudiana), cyclamate (e.g., sodium cyclamate), xylitol, sorbitol, and aspartame.

Flavoring Agents

The antibacterial activity of chlorhexidine can also be reduced if it reacts with compounds such as flavoring agents. However, flavoring agents that do not significantly decrease the antibacterial activity of chlorhexidine compositions have been identified. Accordingly, in one embodiment, the compositions of the invention comprise one or more flavoring agents that do not significantly decrease the antibacterial activity of the oral composition. Suitable flavoring agents include Tutti-Frutti, and Root Beer flavors (e.g., supplied by Medisca, Inc., 661 Route #3, Unit C, Plattsburg, N.Y. 12901. Telephone 1-800-932-1039).

Additionally, it has unexpectedly been discovered that the addition of certain flavoring agents to chlorhexidine containing compositions actually improves the antibacterial activity of the compositions. Thus, in a preferred embodiment, the invention provides a chlorhexidine-containing composition that comprises a flavoring agent that increases the antibacterial activity of the composition as compared to the antibacterial activity of the same composition lacking that flavoring agent. One such flavoring agent is anhydrous raspberry flavor (Product #R1479P available from Spectrum Pharmacy Products, Division of Spectrum Laboratory Products, Inc., 14422 S. San Pedro Street, Gardena, Calif. 90248. Telephone 1-800-791-3210). In one embodiment of the invention, the flavoring agent increases the antibacterial activity of the composition as compared to the antibacterial activity of the same composition lacking that flavoring agent by at least about 10%. In another embodiment of the invention, the flavoring agent increases the antibacterial activity of the composition as compared to the antibacterial activity of the same composition lacking that flavoring agent by at least about 30%. In another embodiment of the invention, the flavoring agent increases the antibacterial activity of the composition as compared to the antibacterial activity of the same composition lacking that flavoring agent by at least about 2-fold.

In some embodiments of the invention, the flavoring agent includes any one of the following flavoring agents, or combinations thereof: mint (e.g., spearmint, peppermint, wintergreen, creme de menthe), strawberry, cherry, tutti frutti, root beer, raspberry (e.g., anhydrous raspberry), and orange.

In some embodiments of the invention, the concentration (W/W) of the flavoring agent (e.g., anhydrous raspberry flavor) is within the range of about 25%-200% of a concentration of a flavoring agent used herein. In some embodiments of the invention, the concentration (W/W) of the flavoring agent is about 50% of a concentration of a flavoring agent used herein. In some embodiments of the invention, the concentration (W/W) of the flavoring agent is within the range of about 50%-100% of a concentration of a flavoring agent used herein. For example, in some embodiments of the invention, the concentration (W/W) of the flavoring agent is about 1.4%-1.5%; in some embodiments, the concentration (W/W) of the flavoring agent is about 0.75%; in some embodiments the concentration (W/W) of the flavoring agent is within the range of from about 0.375% to about 3%; in some embodiments, the concentration (W/W) of the flavoring agent is within the range of from about 0.75% to about 1.5%.

Also provided is a method to promote an antibacterial effect in an animal comprising administering to the animal a composition comprising an effective antibacterial amount of a flavoring and/or a sweetening agent (e.g. anhydrous raspberry flavor), as well as the use of a flavoring and/or a sweetening agent to prepare a medicament useful to promote an antibacterial effect in an animal.

Also provided is a method to prevent or treat dental caries in an animal comprising administering to the animal a composition comprising an effective amount of a flavoring and/or sweetening agent (e.g., anhydrous raspberry flavor), as well as the use of a flavoring and/or sweetening agent to prepare a medicament useful to prevent or treat dental caries in an animal.

Surfactants

Surfactants are often used in commercial chlorhexidine products to facilitate the addition of flavoring agents to the product. Many commercial surfactants chemically inactivate the chlorhexidine. (See Barkovoll, P, Rolla, G, Svendsen, K., Interaction between chlorhexidine digluconate and sodium lauryl sulfate in vivo, J Clin. Periodontol, 1989 October; 16(9):593-5.) The addition of lipophilic flavoring agents (e.g., peppermint oil, etc.) to chlorhexidine solutions will usually result in a cloudy solution. Typically, opaque solutions of chlorhexidine have decreased antimicrobial activity. The compositions of the invention can optionally include one or more surfactants. In one embodiment of the invention, the compositions do not comprise any surfactants. In another embodiment of the invention the compositions do not comprise any non-ionic surfactants.

Chlorhexidine

Chlorhexidine is an antiseptic and disinfectant effective against a wide variety of gram-positive and gram-negative bacteria, fingi, yeast and select viruses. Chlorhexidine has been used since 1959 and is widely available throughout the world. Chemically, chlorhexidine is a strong base and is most stable in its salt forms. Chlorhexidine gluconate (1,1′-hexamethylene bis [5-(p-chlorophenyl biguanide]di-D-gluconate), also known as chlorhexidine digluconate, is a salt formed from chlorhexidine and gluconic acid.

Chlorhexidine salts are adsorbed onto the cell walls of microorganisms, resulting in disruption of the cell wall integrity and leakage of intracellular contents. At low concentrations, chlorhexidine is a bacteriostatic agent, and at higher concentrations it becomes bacteriocidal. A primary benefit of chlorhexidine is its ability to kill bacteria on contact and remain non-toxic to mammalian cells.

Chlorhexidine salts are cationic, which facilitates their adsorption onto the surfaces of the oral mucosa, teeth and plaque, all of which have a net negative charge. The adsorbed chlorhexidine is gradually released from these tissues by diffusion. Thus, chlorhexidine has a substantial residual effect in that it retards microbial growth in the mouth for prolonged periods after application, allowing for interval use rather than daily application.

Chlorhexidine marketed for use in the oral cavity has been in many forms, including mouthwashes (usually 0.1-0.2%), a 1% dental gel, 2% topical oral drops, lozenges, implantable chips, etc. In many countries, these preparations are sold over-the-counter. In the United States, chlorhexidine gluconate is available via prescription and over-the-counter (OTC). Chlorhexidine is available OTC as a topical antiseptic and germicide for use as a surgical scrub, skin cleanser and for preoperative showering or bathing (Drug Facts and Comparisons, 1999).

In the U.S., chlorhexidine for dental use is limited to prescription status and is available as an oral rinse and as a 2.5 mg chip (PerioChip®-Astra). The chip contains 2.5 mg of chlorhexidine gluconate in a glycerin and gelatin matrix and is indicated as an adjunct in scaling and root planing procedures for the reduction of pocket depth in patients with adult periodontitis (Drug Facts and Comparisons, 1999). Chlorhexidine gluconate rinse is available in the U.S. as a 0.12% solution (1.2 mg/ml) for the treatment of gingivitis. This commercial rinse is usually flavored with anise or mint and contains 11.6% (23 proof) alcohol by weight. Approximately 30% of chlorhexidine is retained in the oral cavity following rinsing and is slowly released into the oral fluids (Drug Facts and Comparisons, 1999).

Any suitable source of chlorhexidine can be used in the compositions and methods of the invention. Suitable chlorhexidine starting materials include chlorhexidine salts, as they have enhanced stability over the parent chlorhexidine. Chlorhexidine gluconate (also known as chlorhexidine digluconate), is the preferred salt due to its high water solubility. Other possible compounds include chlorhexidine diacetate and chlorhexidine dihydrochloride.

Geometric Dilution

The invention provides improved methods for preparing hydrocolloidal compositions comprising chlorhexidine. It has been discovered that the addition of chlorhexidine to a hydrocolloidal composition comprising a suspending agent (e.g. a cellulose-derivative) using “geometric dilution” techniques provides an improved chlorhexidine containing hydrocolloidal composition as compared to chlorhexidine containing hydrocolloidal compositions prepared using traditional addition techniques.

“Geometric dilution” is a pharmaceutical term which is normally applied to the extemporaneous method of efficiently combining two unequal amounts of a powdered substance to form a homogenous mixture. The concept of geometric dilution centers on the successive addition and blending of equal quantities of materials.

For example, in the extemporaneous blending of 1 gram of substance A with 20 grams of substance B, the most efficient method to insure a homogenous blend of powders would be to initially blend about 1 gram of substance A with about 1 gram of substance B. After this combination is thoroughly triturated (blended), then about 2 grams of substance B would be added to the A-B mixture. Then 4 more grams of substance B would be added and so forth. This successive addition and blending of equal quantities is an efficient method to arrive at a homogenous mixture.

While the concept of geometric dilution is normally used for powders, it has been found that the slow, geometric addition of a stock chlorhexidine solution first to a small amount of sterile water and then to a pre-hydrated suspending agent-water mixture allows for the formation of a commercially acceptable product without the addition of other surfactants or alcohol.

Other Ingredients

The compositions of the invention can also comprise other active and inactive ingredients. For example, the compositions of the invention can also comprise metal salts (e.g. a copper salt, a zinc salt, or a combination thereof). Typically, the cations (e.g., copper and zinc) are present in the compositions in amounts up to about 1% by weight. In some embodiments, the cations are present in the compositions in amounts up to about 0.5% by weight, or up to about 0.1% by weight. Preferably, the cations are present in at least about 0.01% by weight.

General Methods of Screening for Antimicrobial Activity

The ability of chlorhexidine (CHX) oral compositions to act as antimicrobial agents can be determined using methods that are know in the art, or by using Test A or Test B described below.

Test A: Bactericidal Activity

Rinse formulations were assessed for their bactericidal activity in a laboratory bacterial biofilm system. Streptococcus mutans biofilms were grown on slides in Trypticase Soy Broth supplemented with 0.5% Yeast Extract (TSB-YE), 1.0% glucose and 2.0% sucrose, and incubated for 48 hr in 5% CO₂ at 37° C. Biofilms were exposed to the different formulations for 30 seconds with slow stirring. Biofilms were then extensively washed, harvested, and resuspended in fresh media. Samples were spiral-plated on Mitis-Salivarius agar. Numbers of viable bacteria were determined following standard spiral plating methodology. All of the formulations tested exhibited bactericidal activity above a distilled water control (ANOVA, p<0.01).

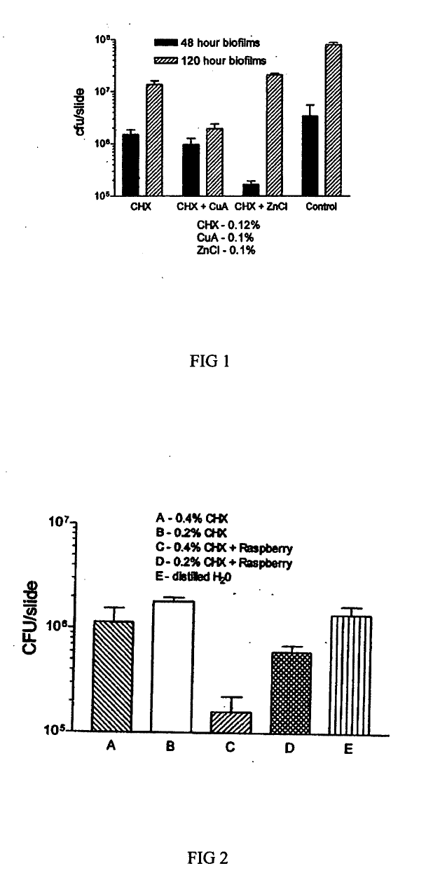

The first series of experiments explored the effect of combinations of chlorhexidine-Cu⁺⁺ and chlorhexidine-Zn⁺⁺ on biofilms of Streptococcus mutans. The chlorhexidine (CHX) used in the experiments depicted in FIG. 1 was made by diluting a chlorhexidine gluconate stock solution (Chlorhexidine gluconate 20% solution, B.P. supplied by Medisca, Inc., 661 Route #3, Unit C, Plattsburg, N.Y. 12901. Telephone 1-800-932-1039) with ultrapure water to form a 0.12% chlorhexidine digluconate solution. This strength mimics the concentration of chlorhexidine gluconate found in commercial rinses available in the United States.

Biofilms of S. mutans were made by immersing slides in Trypticase Soy Broth-Yeast Extract (TSB-YE) supplemented with 1.0% glucose and 2.0% sucrose. Cultures were incubated for 48-120 hours, depending on the density of the biofilm desired, in a 5% CO₂ incubator at 37° C. Biofilms were then exposed to antimicrobial formulations in the following manner. Slides were immersed in the antimicrobial solutions, which were slowly stirring, for a period of 30 seconds. Biofilms were then washed in PBS by repeated dipping through three changes of buffer (10 dips per buffer solution). Biofilms were removed from the slides via scraping with a curette into sterile TSB-YE. Resulting suspensions were vigorously vortexed to disrupt aggregates of cells. These collective procedures typically result in homogeneous suspensions. Numbers of viable cells were determined by standard Spiral-plating methodology. Results from the experiments are shown in FIG. 1.

Exposure of biofilms to chlorhexidine-cation combinations resulted in 1-2 orders of magnitude decreases in number of viable cells over chlorhexidine alone (p<0.05). Earlier studies had shown that the metal ions alone had little to no activity against biofilms. Interestingly, the CHX-Zn⁺⁺ combination was more effective against light biofilms, whereas CHX-Cu⁺⁺ caused greater decreases against more heavy biofilms. The mechanism(s) for this difference is currently unknown. However, these results are reproducible in the laboratory and are one reason for determining the efficacy of chlorhexidine composition formulations with each of the metal cations. It is conceivable that a particular chlorhexidine-cation formulation may exhibit greater activity than another, depending on the amount of supragingival plaque present.

Another series of studies focused on the effect of adding flavoring agents to the chlorhexidine formulations. The chlorhexidine (CHX) used in the experiments depicted in FIG. 2 was made by diluting a chlorhexidine gluconate stock solution (Chlorhexidine gluconate 20% solution, B.P. supplied by Medisca, Inc., 661 Route #3, Unit C, Plattsburg, N.Y. 12901. Telephone 1-800-932-1039) with ultrapure water to form a 0.2% and 0.4% solution. Representative results of these studies are shown in FIG. 2.

The addition of raspberry flavoring to 0.2% chlorhexidine had little effect on bactericidal activity but in fact caused slightly higher levels of killing. In contrast, a significant decrease (p<0.05) in the numbers of viable cells was seen with raspberry added to 0.4% chlorhexidine. Therefore, with this particular flavoring agent, an enhanced level of bactericidal activity was seen over chlorhexidine alone.

Test B

Some oral bacteria are believed to cause cavities by producing acid in the mouth. Therefore, the ability of a composition to prevent and treat cavities can be measured by determining its effects on bacterial acid production.

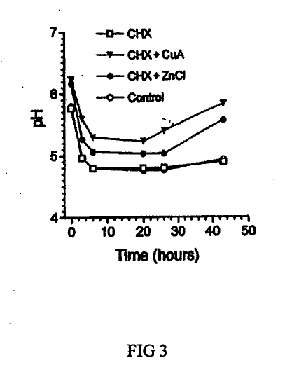

Accordingly, the effect of representative compositions on the ability of S. mutans to produce acid was also assessed. Biofilms were exposed to CHX-Cu⁺⁺ or CHX-Zn⁺⁺ as described, then washed, harvested, and resuspended in sterile TSB-YE supplemented with 1.0% glucose at standardized concentrations. Changes in culture pH were measured over time. Representative results of these studies are illustrated in FIG. 3.

The chlorhexidine (CHX) used in the experiments depicted in FIG. 3 was made by diluting a chlorhexidine gluconate stock solution (Chlorhexidine gluconate 20% solution, B.P. supplied by Medisca, Inc., 661 Route #3, Unit C, Plattsburg, N.Y. 12901. Telephone 1-800-932-1039) with ultrapure water to form a 0.12% chlorhexidine digluconate solution. This strength mimics the concentration of chlorhexidine gluconate found in commercial rinses (Peridex®—Zila Pharmaceuticals 5227 N. 7^{th} Street, Phoenix, Ariz. 85014. Telephone (602) 266-6700) available in the United States.

Chlorhexidine was at a concentration of 0.06%, and cupric acetate and zinc chloride were at concentrations of 0.1% w/v. Exposure of chlorhexidine alone had little effect on acid production. In sharp contrast, pH curves for the CHX-cation formulations showed less steep decreases in pH as well as more rapid return of the cell suspensions towards neutrality. This latter observation may be in part due to the increased levels of cell killing over the long-term. Nevertheless, these data show that the CHX-cation formulations not only have enhanced bactericidal activity (p<0.05), but also affect the ability of viable cells to produce acid in the presence of sugar. Thus, certain embodiments of the invention provide CHX-cation formulations that affect the ability of viable cells to produce acid, for example, in the presence of sugar. This effect will decrease the formation of cavities.

It has been determined that the chlorhexidine composition formulations exhibit bactericidal activity relative to a chlorhexidine control. Suspensions of S. mutans exposed to the compositions by mixing small aliquots of the composition into the suspensions resulted in complete kill within one hour of incubation: 0.1% composition—0 cfu/ml, Chlorhexidine gluconate (0.12% CHX)—0 cfu/ml, buffer control—10⁶ cfu/ml. These results show that the disclosed chlorhexidine composition formulations exert strong bactericidal activity similar to control.

Certain embodiments of the present invention will now be illustrated by the following non-limiting examples.

### EXAMPLES GENERAL SECTION

The following equipment was used in the Examples hereinbelow: Ointment mill: manufacturer Exakt Appartebau of Germany, model TYP/NO-13992; Balance: manufacturer Denver Instrument Co. U.S.A., model TR-403; Hot plate/magnetic stirrer: manufacturer Fisher Scientific U.S.A., Serial 004n0519.

### Example 1

### Chlorhexidine Gluconate 1.0% Gel (W/W)

A representative composition of the invention was prepared as follows using a method of the invention.

TABLE 1

| INGREDIENT | SOURCE | WEIGHT | |
|---|---|---|---|
| Chlorhexidine gluconate 20% | Spectrum ® | 10.0 | grams |
| solution, B.P. | | | |
| Raspberry flavor (anhydrous) | Spectrum ® | 2.8 | grams |
| Aspartame, N.F. | Spectrum ® | 0.8 | grams |
| Hydroxypropyl methylcellulose 2208, | Spectrum ® | 4.0 | grams |
| 4000 cps, U.S.P. | | | |
| Sterile water for irrigation, U.S.P. | Spectrum ® | qs 200 | grams |
| (Baxter Healthcare ®) | | | |

Compounding Procedure

All utensils and glassware were cleaned with 95% ethanol (supplied by Medisca, Inc., 661 Route #3, Unit C, Plattsburg, N.Y. 12901) and rinsed with sterile water (Sterile water for irrigation, U.S.P.-Baxter Healthcare®). Sterile water (125 g) and a stir bar were added to a 400 ml Pyrex® beaker and the beaker was placed on a hot plate/magnetic stirrer (stir setting 5). The water was heated at heat setting 5 to 50° C. and the heat source was turned off. Aspartame (0.8 g) (Aspartame, N.F.-Spectrum®) was mixed into the heated water at stir setting 5 until the aspartame dissolved. HPMC (4.0 g) (Hydroxypropyl methylcellulose 2208, 4000 cps, U.S.P-Spectrum®) was added slowly to the vortex over the course of 10 minutes and the resulting mixture was stirred for 5 additional minutes or until hydrated. In a second beaker, CHX 20% solution (10 g) (Chlorhexidine gluconate 20% solution, B.P-Spectrum®) was added slowly to an equal amount of sterile water with stirring. An additional 10 grams of sterile water was added with stirring. The HPMC mixture was cooled to room temperature (about 22° C.), and the CHX-water solution was mixed into the HPMC dropwise by geometric dilution at a stir setting of 5, without heating. Raspberry flavoring (2.8 g) (Raspberry flavor (anhydrous)-Spectrum®) was added dropwise to the center of the vortex and stirring was continued for one additional minute after the flavoring was added. Slowly, the remaining sterile water QS (quantity sufficient) to 200 grams total composition weight was added and mixing was continued for 5 minutes on stir setting 5. The resulting composition was placed in an amber plastic oval and was sealed for 24 hours to allow for hydration. The composition was processed through an ointment mill until smooth, placed in an amber plastic oval, sealed tightly, and stored at controlled room temperature (20-25° C./68-77° F.).

### Example 2

### Chlorhexidine Gluconate 1.0% Gel with 0.29% Zinc Ion (W/W)

Another representative composition of the invention was prepared as follows using a method of the invention.

TABLE 2

| INGREDIENT | SOURCE | WEIGHT | |
|---|---|---|---|
| Chlorhexidine gluconate 20% | Spectrum ® | 12.5 | grams |
| solution, B.P. | | | |
| Zinc gluconate, U.S.P. | Spectrum ® | 5 | grams |
| Raspberry flavor (anhydrous) | Spectrum ® | 3.5 | grams |
| Aspartame, N.F. | Spectrum ® | 1.0 | gram |
| Hydroxypropyl methylcellulose 2208, | Spectrum ® | 5.0 | grams |
| 4000 cps, U.S.P. | | | |
| Sterile water for irrigation, U.S.P. | Spectrum ® | 223.0 | grams |
| (Baxter Healthcare ®) | | | |

Compounding directions:
1. Clean all utensils and glassware with 95% ethanol (supplied by Medisca, Inc., 661 Route #3, Unit C, Plattsburg, N.Y. 12901) and rinse with sterile water (Sterile water for irrigation, U.S.P.-Baxter Healthcare®)
2. Weigh out all ingredients.
3. Add sterile water (180 g) to 400 ml Pyrex® beaker. Add stir bar and set on stir setting 5.
4. Heat on heat setting 5 until 50° C. Turn off heat source.
5. Mix aspartame (1 g) (Aspartame, N.F.-Spectrum®) into heated water, stirring on setting 5 until aspartame is dissolved.
6. Add HPMC (5.0 g) (Hydroxypropyl methylcellulose2208, 4000 cps, U.S.P-Spectrum®) slowly to vortex over the course of 10 minutes. Once added continue stirring for 5 minutes or until hydrated
7. In a second beaker, add CHX 20% solution (12.5 g) (Chlorhexidine gluconate 20% solution, B.P-Spectrum®) slowly to an equal amount of sterile water with stirring. Add an additional 10 grams of sterile water with stirring.
8. Once HPMC mixture has cooled to room temperature (22° C.), mix the CHX-water solution into the HPMC by geometric dilution. Stir setting 5, no heat.
9. Dissolve zinc gluconate (5 g) (zinc gluconate, U.S.P.-Spectrum®) in 10 cc of sterile water add to HPMC mixture slowly with stirring.
10. Add raspberry flavoring (3.5 g) (Raspberry flavor (anhydrous)-Spectrum®) dropwise to center of vortex with stirring. Mix 1 additional minute after adding the flavoring.
11. Slowly add remaining sterile water QS (quantity sufficient) to bring the weight of the composition up to 250 grams. Continue mixing for 5 minutes on stir setting 5.
12. The composition was placed in an amber plastic oval and was sealed for 24 hours to allow for hydration. The composition was then processed through an ointment mill (Ointment mill—manufacturer Exakt Appartebau of Germany, model TYP/NO-13992) until smooth, placed in an amber plastic oval, sealed tightly, and stored at controlled room temperature (20-25° C./68-77° F.).

### Example 3

### Chlorhexidine Gluconate 1.0% Gel with 0.14% Copper Ion (W/W)

Another representative composition of the invention was prepared as follows using a method of the invention.

TABLE 3

| INGREDIENT | SOURCE | WEIGHT | |
|---|---|---|---|
| Chlorhexidine gluconate 20% | Spectrum ® | 12.5 | grams |
| solution, B.P. | | | |
| Copper gluconate, U.S.P. | Spectrum ® | 2.5 | grams |
| Raspberry flavor (anhydrous) | Spectrum ® | 3.5 | grams |
| Aspartame, N.F. | Spectrum ® | 1.0 | gram |
| Hydroxypropyl methylcellulose 2208, | Spectrum ® | 5.0 | grams |
| 4000 cps, U.S.P. | | | |
| Sterile water for irrigation, U.S.P. | Spectrum ® | 225.5 | grams |
| (Baxter Healthcare ®) | | | |

Compounding directions:
1. Clean all utensils and glassware with 95% ethanol (supplied by Medisca, Inc., 661 Route #3, Unit C, Plattsburg, N.Y. 12901.

Telephone 1-800-932-1039) and rinse with sterile water (Sterile water for irrigation, U.S.P.-Baxter Healthcare®)
2. Weigh out all ingredients.
3. Add sterile water (180 g) to 400 ml Pyrex® beaker. Add stir bar and set on stir setting 5.
4. Heat on heat setting 5 until 50° C. Turn off heat source.
5. Mix aspartame (1 g) (Aspartame, N.F.-Spectrum®) into heated water, stirring on setting 5 until aspartame is dissolved.
6. Add HPMC (5.0 g) (Hydroxypropyl methylcellulose2208, 4000 cps, U.S.P-Spectrum®) slowly to vortex over the course of 10 minutes. Once added continue stirring for 5 minutes or until hydrated
7. In a second beaker, add CHX 20% solution (12.5 g) (Chlorhexidine gluconate 20% solution, B.P-Spectrum®).slowly to an equal amount of sterile water with stirring. Add an additional 10 grams of sterile water with stirring.
8. Once HPMC mixture has cooled to room temperature (22° C.), mix the CHX-water solution into the HPMC by geometric dilution. Stir setting 5, no heat.
9. Dissolve copper gluconate (2.5 g) (copper gluconate, U.S.P.-Spectrum®) in 10 cc of sterile water and stir until dissolved.
10. Add raspberry flavoring (Raspberry flavor (anhydrous)-Spectrum®) dropwise to center of vortex with stirring. Mix 1 additional minute after adding the flavoring.
11. Slowly add remaining sterile water QS (quantity sufficient) to 250 grams. Continue mixing for 5 minutes on stir setting 5.
12. The composition was placed in an amber plastic oval and was sealed for 24 hours to allow for hydration. The composition was then processed through an ointment mill (Ointment mill—manufacturer Exakt Appartebau of Germany, model TYP/NO-13992) until smooth, placed in an amber plastic oval, sealed tightly, and stored at controlled room temperature (20-25° C./68-77° F.).

### Example 4

### Chlorhexidine Gluconate 1.0% Gel with 0.07% Copper Ion (W/W)

Another representative composition of the invention was prepared as follows using a method of the invention.

TABLE 4

| INGREDIENT | SOURCE | WEIGHT | |
|---|---|---|---|
| Chlorhexidine gluconate 20% | Spectrum ® | 20.0 | grams |
| solution, B.P. | | | |
| Copper gluconate, U.S.P. | Spectrum ® | 2.0 | grams |
| Raspberry flavor (anhydrous) | Spectrum ® | 5.6 | grams |
| Aspartame powder, N.F. | Spectrum ® | 1.6 | gram |
| Hydroxypropyl methylcellulose 2208, | Spectrum ® | 8.0 | grams |
| 4000 cps, U.S.P. | | | |
| Sterile water for irrigation, U.S.P. | Spectrum ® | QS to 400 | g |
| (Baxter Healthcare ®) | | | |

Compounding directions:
1. Clean all utensils and glassware with 95% ethanol (supplied by Medisca, Inc., 661 Route #3, Unit C, Plattsburg, N.Y. 12901. Telephone 1-800-932-1039) and rinse with sterile water (Sterile water for irrigation, U.S.P.-Baxter Healthcare®)
2. Weigh out all ingredients.
3. Add sterile water (280 g) to 600 ml Pyrex® beaker. Add stir bar and set on stir setting 5.
4. Heat on heat setting 5 until 50° C. Turn off heat source.
5. Mix aspartame (1.6 g) (Aspartame, N.F.-Spectrum®) into heated water, stirring on setting 5 until aspartame is dissolved.
6. Add copper gluconate (2.0 g) (copper gluconate, U.S.P.-Spectrum®) and stir until dissolved.
7. Add HPMC (8.0 g) (Hydroxypropyl methylcellulose2208, 4000 cps, U.S.P-Spectrum®) slowly to vortex over the course of 10 minutes. Once added continue stirring for 5 minutes or until hydrated
8. In a second beaker, add CHX 20% solution (20.0 g) (Chlorhexidine gluconate 20% solution, B.P-Spectrum®).slowly to an equal amount of sterile water with stirring. Add an additional 10 grams of sterile water with stirring.
9. Once HPMC mixture has cooled to room temperature (22° C.), mix the CHX-water solution into the HPMC by geometric dilution. Stir setting 5, no heat.
10. Add raspberry flavoring (5.6 g) (Raspberry flavor (anhydrous)-Spectrum®) dropwise to center of vortex with stirring. Mix 1 additional minute after adding the flavoring.
11. Slowly add remaining sterile water QS (quantity sufficient) to bring the total composition weight to 400 grams. Continue mixing for 5 minutes on stir setting 5.
12. The composition was placed in an amber plastic oval and was sealed for 24 hours to allow for hydration. The composition was then processed through an ointment mill (Ointment mill—manufacturer Exakt Appartebau of Germany, model TYP/NO-13992) until smooth, placed in an amber plastic oval, sealed tightly, and stored at controlled room temperature (20-25° C./68-77° F.).

### Example 5

### Clinical Trial

For most children living in the United States, tooth decay is no longer a serious problem. For a surprising number of low income and minority children, however, tooth decay is a problem that interferes with their ability to eat, speak, sleep, play, grow and learn. At a time when dental decay rates have been declining for the general population, the decay rates for children living in poverty have remained high or actually increased. National surveys of 3, 4 and 5 year old children attending Head Start Preschool Programs find rates of dental decay ranging from 50-85%. However, efforts to prevent tooth decay in high risk groups of children through education, dental visits and fluoride applications have largely failed. This Example describes the efficacy of a 1% chlorhexidine gel in reducing levels of cariogenic bacteria in children at high-risk for decay.

Several goals of the study described herein were: to determine how different application intervals for chlorhexidine gel application affect the levels of decay-causing oral bacteria (Streptococcus mutans) over time; to assess the efficacy of a 1.0% chlorhexidine gel in preventing tooth decay in the primary dentition of high-risk children using different application intervals; and to assess the feasibility and practicality of implementing preventive dental programs using chlorhexidine gel in community-based settings serving high-risk young children.

The antibacterial agent used was a raspberry-flavored 1% chlorhexidine gel formulated. This chlorhexidine gel formulation is about 10 times stronger than the commercially available 0.12% chlorhexidine rinse currently available in the United States. Further, this gel formulation makes application on the teeth of preschool-aged children possible. In addition, whereas commercially available chlorhexidine products are typically bad tasting, it was found that the raspberry-flavored chlorhexidine gel formulation is superior in taste and was acceptable to young children.

Methods

The participants in this study were 3, 4 and 5 year old children attending 17 Head Start preschool programs in Muscatine and other East Central Iowa communities.

Each participating Head Start classroom was assigned a specific chlorhexidine gel regimen (interval) to be tested. The regimens to be tested included: one application of active gel (i.e., chlorhexidine gel) each month for nine months; and one application of placebo gel (i.e., gel without chlorhexidine) each month for nine months.

Within each classroom, participating children were assigned randomly to either the active treatment group or a placebo control group. Children participating in the placebo control groups received the same interval of treatment as their classmates but with a gel containing no chlorhexidine. Assignment to treatment groups based on this study design resulted in two discrete treatment groups as follows: the treatment protocol for Group 1 included a one-minute toothbrush application of 1.0 cc chlorhexidine gel each month for nine months, for a total of 9 treatments; the treatment protocol for Group 2 included a one-minute toothbrush application of 1.0 cc placebo gel each month for nine months for a total of 9 treatments.

Following completion of initial clinical exams, the project coordinator traveled to each Head Start classroom for sampling of Streptococcus mutans bacteria and for application of the chlorhexidine gel or placebo gel. Streptococcus mutans was sampled immediately prior to each application of either chlorhexidine or placebo gel. The sampling procedure for Streptococcus mutans consisted of swabbing the teeth and gums of the children with a sterile cotton tip applicator until an adequate plaque sample has been obtained. The cotton tip applicators were then placed in individually labeled tubes of transport media. Samples were then processed and analyzed.

Results

The primary outcome variable of this clinical study was the level of the cariogenic bacteria Streptococcus mutans in the plaque of the children. It has been firmly established that these organisms are the primary etiological agent of dental decay. Therefore, treatment regimens that reduce the counts of these bacteria in these children will substantially reduce their risk of future dental decay.

A secondary outcome measure was decay rates in the study children. While a 9-month time period is sometimes not sufficient to detect differences in decay rates among children, it is believed that this period should be sufficient as the group of children enrolled in this study were at higher risk for dental decay than the general public, and the research team is experienced in identifying carious lesions in pre-school aged children at very early stages.

A third outcome measure, observational in nature, related to the feasibility and logistics of implementing a preventive dental program involving multiple applications of chlorhexidine gel in a community-based setting.

A summary of the effect of the chlorhexidine gel on levels of cariogenic bacteria in these children has been generated. Data analysis was restricted to subjects who were present for at least 5 of 7 possible gel applications (n=215). For subjects with high levels of Streptococcus mutans at baseline, 70.6% of those receiving a placebo gel remained at high levels at study completion as compared to 29.2% of those receiving chlorhexidine (p=0.039).

Thus, monthly applications, e.g., by toothbrush, of 1% chlorhexidine gel lowers Streptococcus mutans levels for high risk children and thus reduces a major risk factor for the development of caries.

### Example 6

### Stability of Sweetened Chlorhexidine Compositions

The stability of sweetened hydrocolloidal chlorhexidine compositions, with and without added copper or zinc ions, was examined. The composition potency was determined by HPLC. The HPLC system included of a UV detector, pump, integrator, autoinjector with 10 μL Loop, and a HPLC column. The mobile phase consisted of acetonitrile and an aqueous solution containing phosphate buffer and tirethylamine. The analysis conditions were: 1.0 mL/min flow rate, ambient temperature column, and analytical wavelength of 238 nm. USP system suitability and accuracy protocols were carried out. Additional instrumentation used for sample and standards preparation included an analytical balance, a pH meter and filters (solvent and sample).

Samples of the sweetened hydrocolloidal chlorhexidine compositions, with and without added copper or zinc ions, were stored at controlled room temperature. (Refer to Examples 1, 2, 3, and 4 for composition details.) Periodically, aliquots were removed from the compositions and analyzed for chlorhexidine potency. Analyses were carried out as follows: 1 gram aliquot of gel was weighed into a 10 ml volumetric flask which was qs with mobile phase and filtered. 1.0 mL of the resultant solution was transferred into a 50 ml volumetric flask and qs with mobile phase. Appropriate standards were selected to include concentrations that covered the range of sample concentrations. Samples and standards were loaded into the autoinjector using a random array. Each gel was assayed in triplicate for chlorhexidine potency.

As is depicted in Table 5, addition of copper or zinc ions to the chlorhexidine preparations improves the stability, and shelf-life, of the chlorhexidine preparations. Thus, certain embodiments of the present invention provide chlorhexidine compositions, e.g., sweetened chlorhexidine compositions, having improved stability and shelf-life. These chlorhexidine compositions include a copper salt, a zinc salt, or a combination thereof.

TABLE 5
Stability Determinations for 1% Chlorhexidine Compositions

| | | | % of Initial | |
|---|---|---|---|---|
| | Storage | Age | Potency | Standard |
| Composition | Temp. | (Months) | Assay | Deviation* |
| No added | 25° C. | 0 | 100 | 1.2 |
| metal ions¹ | | 4 | 93.3 | 0.33 |
| | | 12 | 93.9 | 0.59 |
| 0.29% (w/w) | 25° C. | 0 | 100 | 0.27 |
| Zinc ions² | | 4 | 101.5 | 0.29 |
| | | 12 | 99.9 | 1.55 |
| 0.14% (w/w) | 25° C. | 0 | 100 | 0.42 |
| Copper ions³ | | 4 | 106.8 | 0.89 |
| | | 12 | 97.6 | 1.1 |
| 0.07% (w/w) | 25° C. | 0 | 100 | 1.23 |
| Copper ions⁴ | | 4 | 99.3 | 0.42 |
| | | 12 | 94.6 | 1.1 |

^{1,2,3,4}Refer to Examples 1, 2, 3, and 4, respectively, for composition details.
*Relative standard deviation of triplicate chlorhexidine potency assay (%).

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the below enumerated embodiments of the invention.

1. An alcohol-free hydrocolloidal chlorhexidine composition.

2. The composition of claim 1 that comprises chlorhexidine gluconate.

3. The composition of claim 1 that comprises chlorhexidine diacetate or chlorhexidine dihydrochloride.

4. The composition of claim 1 that comprises a sweetening agent.

5. The composition of claim 4, wherein the sweetening agent does not significantly reduce the antibacterial activity of the composition.

6. The composition of claim 4, wherein the sweetening agent is selected from the group consisting of aspartame, sorbitol, xylitol, and sucralose.

7. The composition of claim 4, wherein the sweetening agent is aspartame.

8. The composition of claim 4, wherein the sweetening agent is not sorbitol.

9. The composition of claim 1 that comprises a flavoring agent.

10. The composition of claim 9, wherein the flavoring agent is a raspberry flavoring agent.

11. The composition of claim 1 that comprises a copper salt, a zinc salt, or a combination thereof.

12. The composition of claim 11, wherein copper and/or zinc ion is present in at least about 0.05% w/w.

13. The composition of claim 1 that comprises cellulose or a cellulose-derivative.

14. The composition of claim 13 that comprises hydroxypropyl methylcellulose, hydroxyethyl cellulose, or hydroxypropyl cellulose, or a combination thereof.

15. The composition of claim 13 that comprises hydroxypropyl methylcellulose.

16. A hydrocolloidal composition comprising chlorhexidine and a flavoring agent that does not significantly reduce the antibacterial activity of the composition.

17. The composition of claim 16 that comprises chlorhexidine gluconate.

18. The composition of claim 16 that comprises chlorhexidine diacetate or chlorhexidine dihydrochloride.

19. The composition of claim 16 that comprises a sweetening agent.

20. The composition of claim 19 wherein the sweetening agent does not significantly reduce the antibacterial activity of the composition.

21. The composition of claim 19, wherein the sweetening agent is selected from the group consisting of aspartame, sorbitol, xylitol, and sucralose.

22. The composition of claim 19 wherein the sweetening agent is aspartame.

23. The composition of claim 19, wherein the sweetening agent is not sorbitol.

24. The composition of claim 16 wherein the flavoring agent is a raspberry flavoring agent.

25. The composition of claim 16 that comprises a copper salt, a zinc salt, or a combination thereof.

26. The composition of claim 25, wherein copper and/or zinc ion is present in at least about 0.05% w/w.

27. The composition of claim 16 that comprises cellulose or a cellulose-derivative.

28. The composition of claim 27 that comprises hydroxypropyl methylcellulose, hydroxyethyl cellulose, or hydroxypropyl cellulose, or a combination thereof.

29. The composition of claim 27 that comprises hydroxypropyl methylcellulose.

30. A hydrocolloidal composition comprising chlorhexidine and a sweetening agent that does not significantly reduce the antibacterial activity of the oral composition.

31. The composition of claim 30 that comprises chlorhexidine gluconate.

32. The composition of claim 30 that comprises chlorhexidine diacetate or chlorhexidine dihydrochloride.

33. The composition of claim 30 wherein the sweetening agent is selected from the group consisting of aspartame, sorbitol, xylitol, and sucralose.

34. The composition of claim 33 wherein the sweetening agent is aspartame.

35. The composition of claim 30 wherein the sweetening agent is not sorbitol.

36. The composition of claim 30 that comprises a flavoring agent.

37. The composition of claim 36 wherein the flavoring agent is a raspberry flavoring agent.

38. The composition of claim 30 that comprises a copper salt, a zinc salt, or a combination thereof.

39. The composition of claim 30 which does not comprise a surfactant.

40. The composition of claim 30 that comprises cellulose or a cellulose-derivative.

41. The composition of claim 40 that comprises hydroxypropyl methylcellulose, hydroxyethyl cellulose, or hydroxypropyl cellulose, or a combination thereof.

42. The composition of claim 40 that comprises hydroxypropyl methylcellulose.

43. An oral composition comprising chlorhexidine and a flavoring agent that increases the antibacterial activity of the oral composition.

44. The composition of claim 43 that comprises chlorhexidine gluconate.

45. The composition of claim 44 that comprises chlorhexidine diacetate or chlorhexidine dihydrochloride.

46. The composition of claim 43 that comprises a sweetening agent.

47. The composition of claim 46, wherein the sweetening agent is not sorbitol.

48. The composition of claim 46, wherein the sweetening agent is selected from the group consisting of aspartame, sorbitol, xylitol, and sucralose.

49. The composition of claim 46 wherein the sweetening agent is aspartame.

50. The composition of claim 43 which is a hydrocolloidal composition.

51. The composition of claim 43 wherein the flavoring agent is a raspberry flavoring agent.

52. The composition of claim 43 that comprises a copper salt, a zinc salt, or a combination thereof.

53. The composition of claim 52, wherein copper and/or zinc ion is present in at least about 0.05% w/w.

54. The composition of claim 43 that comprises cellulose or a cellulose-derivative.

55. The composition of claim 54 that comprises hydroxypropyl methylcellulose, hydroxyethyl cellulose, or hydroxypropyl cellulose, or a combination thereof.

56. The composition of claim 54 that comprises hydroxypropyl methylcellulose.

57. A method for preparing a hydrocolloidal chlorhexidine composition comprising adding a solution containing chlorhexidine or a salt thereof to a hydrocolloidal suspension to provide the hydrocolloidal chlorhexidine composition.

58. The method of claim 57, wherein the hydrocolloidal chlorhexidine composition is alcohol-free.

59. The method of claim 57, wherein the solution containing chlorhexidine comprises water.

60. The method of claim 57, wherein the solution containing chlorhexidine is chlorhexidine in water.

61. The method of claim 57 wherein the solution containing chlorhexidine is added to the hydrocolloidal suspension dropwise.

62. The method of claim 57 wherein the solution containing chlorhexidine and the hydrocolloidal suspension are combined by geometric dilution.

63. The method of claim 57, wherein the hydrocolloidal suspension is prepared from water and one or more suspending agents.

64. The method of claim 57 wherein the hydrocolloidal suspension that comprises chlorhexidine gluconate.

65. The method of claim 57 wherein the hydrocolloidal suspension comprises chlorhexidine diacetate or chlorhexidine dihydrochloride.

66. The method of claim 57 wherein the hydrocolloidal suspension comprises a sweetening agent.

67. The method of claim 66 wherein the sweetening agent does not significantly reduce the antibacterial activity of the composition.

68. The method of claim 66, wherein the sweetening agent is selected from the group consisting of aspartame, sorbitol, xylitol, and sucralose.

69. The method of claim 66 wherein the sweetening agent is aspartame.

70. The method of claim 66, wherein the sweetening agent is not sorbitol.

71. The method of claim 57 further comprising adding a flavoring agent to the hydrocolloidal chlorhexidine composition.

72. The method of claim 71 wherein the flavoring agent is a raspberry flavoring agent.

73. The method of claim 57 further comprising adding a copper salt, a zinc salt, or a combination thereof, to the hydrocolloidal chlorhexidine composition.

74. The method of claim 73, wherein at least about 0.05% w/w of copper and/or zinc ion is added to the hydrocolloidal chlorhexidine composition.

75. The method of claim 57 wherein the hydrocolloidal suspension comprises cellulose or a cellulose-derivative.

76. The method claim 75 wherein the hydrocolloidal suspension comprises hydroxypropyl methylcellulose, hydroxyethyl cellulose, or hydroxypropyl cellulose, or a combination thereof.

77. The method of claim 75 wherein the hydrocolloidal suspension comprises hydroxypropyl methylcellulose.

78. A method for preparing an improved composition comprising chlorhexidine comprising combining a flavoring agent that increases the antibacterial activity with a composition comprising chlorhexidine.

79. The method of claim 78 wherein the flavoring agent is a raspberry flavoring agent.

80. The method of claim 78 wherein the composition comprising chlorhexidine is formulated for oral administration to an animal.

81. The method of claim 78 wherein the composition comprising chlorhexidine is a hydrocolloidal composition.

82. The method of claim 78 wherein the composition comprising chlorhexidine is alcohol-free.

83. The method of claim 78 wherein the improved composition comprising chlorhexidine is alcohol-free.

84. The method of claim 78 wherein the composition comprising chlorhexidine comprises chlorhexidine gluconate.

85. The method of claim 78 wherein the composition comprising chlorhexidine comprises chlorhexidine diacetate or chlorhexidine dihydrochloride.

86. The method of claim 78, wherein the composition comprising chlorhexidine is a hydrocolloidal suspension comprising one or more suspending agents.

87. The method of claim 78 wherein the composition comprising chlorhexidine comprises a sweetening agent.

88. The method of claim 87 wherein the sweetening agent does not significantly reduce the antibacterial activity of the composition.

89. The method of claim 87, wherein the sweetening agent is selected from the group consisting of aspartame, sorbitol, xylitol, and sucralose.

90. The method of claim 87 wherein the sweetening agent is aspartame.

91. The method of claim 87, wherein the sweetening agent is not sorbitol.

92. The method of claim 78 wherein the improved composition comprising chlorhexidine comprises a copper salt, a zinc salt, or a combination thereof.

93. The method of claim 92, wherein the improved composition comprising chlorhexidine comprises at least about 0.05% w/w copper and/or zinc ions.

94. The method of claim 78 wherein the composition comprising chlorhexidine comprises cellulose or a cellulose-derivative.

95. The method of claim 78 wherein the composition comprising chlorhexidine comprises hydroxypropyl methylcellulose, hydroxyethyl cellulose, or hydroxypropyl cellulose, or a combination thereof.

96. The method of claim 78 wherein the composition comprising chlorhexidine comprises hydroxypropyl methylcellulose.

97. A method comprising:
mixing a sweetening agent into heated water at about 50° C. with stirring to provide a mixture;
allowing the mixture to cool to about 20-25° C.;
slowly adding hydroxypropyl methylcellulose to the mixture with stirring;
adding a solution of chlorhexidine to the mixture dropwise by geometric dilution with stirring to provide a chlorhexidine mixture;
optionally adding a copper salt, a zinc salt, or a combination thereof, with stirring to the chlorhexidine mixture;
optionally adding a raspberry flavoring agent with stirring to the chlorhexidine mixture; and
optionally diluting the mixture with sterile water;
to produce an oral composition containing chlorhexidine.

98. The method of claim 96, wherein the sweetening agent is selected from the group consisting of aspartame, sorbitol, xylitol, and sucralose.

99. The method of claim 96, wherein the sweetening agent is aspartame.

100. The method of claim 96, wherein the sweetening agent is not sorbitol.

101. The method of claim 97 wherein the aqueous solution of chlorhexidine is added to the mixture at a rate of about one drop per second.

102. A composition prepared by the method described in claim 57.

103. A method to promote an antibacterial effect comprising administering an effective amount of a composition as described in claim 1.

104. The method of claim 103 wherein the composition is administered to the mouth of the animal.

105. A method to prevent or treat dental caries comprising administering an effective amount of a composition as described in claim 1.

106. A composition as described in claim 1.

107. The use of a composition as described in claim 1 to prepare a medicament useful to promote to an antibacterial effect in an animal.

108. The use of a composition as described in claim 1 to prepare a medicament useful to prevent or treat dental caries in an animal.

109. The use of claim 107 wherein the animal is a human.
